(19) United States
(12) Patent Application Publication
(10) Pub. No.: US 2022/0036007 A1
(43) Pub. Date: Feb. 3, 2022

(54) BOOTSTRAPPING RELATION TRAINING DATA

(71) Applicant: INTERNATIONAL BUSINESS MACHINES CORPORATION, Armonk, NY (US)

(72) Inventors: Pai-Fang Hsiao, Dublin (IE); Scott Carrier, New Hill, NC (US)

(21) Appl. No.: 16/944,396

(22) Filed: Jul. 31, 2020

(51) Int. Cl. G06F 40/295; G06N 20/00; G06N 5/04; G16H 10/60; G06F 40/169; G06F 40/284; G06F 40/30; G06F 40/247

(52) U.S. Cl. CPC G06F 40/295; G06N 20/00; G06N 5/04; G16H 10/60; G06F 40/205; G06F 40/284; G06F 40/30; G06F 40/247; G06F 40/169

(57) ABSTRACT

Aspects of the invention include a computer-implemented method for bootstrapping relation training data. The method includes traversing a corpus to detect a first passage having a first set of co-occurring entities and intervening tokens associated with a relation type. Identifying a first predicate frame of the first passage based on the co-occurring entities and intervening tokens. Traversing the corpus again to detect a second passage having a second predicate frame with a same semantic structure as the first predicate frame, wherein the passage contains a second set of co-occurring entities associated with the relation during first instance that the processor did not detect during the first time. Detecting a second set of co-occurring entities in the second passage based on the second predicate frame. Annotating the second set of co-occurring entities to have a same relation as the first set of co-occurring entities.

### BACKGROUND

The present invention generally relates to programmable computing systems, and more specifically, to bootstrapping relation training data.

Computer information systems can receive search queries from a user and provide answers back to the user. In information retrieval, a system is tasked with automatically answering a question posed in natural language to the system. The information retrieval system can retrieve an answer to the search query by searching a data corpus for documents matching the search query. To assist the information retrieval system, the documents are annotated to describe relations between co-occurring entities. The information retrieval system can analyze the annotations as well as keywords in the documents to determine which documents best answer the search query.

### SUMMARY

Embodiments of the present invention are directed to bootstrapping relation data. A non-limiting example computer-implemented method includes traversing a corpus to detect a first passage having a first set of co-occurring entities and intervening tokens associated with a relation type. Identifying a first predicate frame of the first passage based on the co-occurring entities and intervening tokens. Traversing the corpus again to detect a second passage having a second predicate frame with a same semantic structure as the first predicate frame, wherein the passage contains a second set of co-occurring entities associated with the relation during first instance that the processor did not detect during the first time. Detecting a second set of co-occurring entities in the second passage based on the second predicate frame. Annotating the second set of co-occurring entities to have a same relation as the first set of co-occurring entities.

Other embodiments of the present invention implement features of the above-described method in computer systems and computer program products.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 illustrates a block diagram of a system for bootstrapping relation training data in accordance with one or more embodiments of the present invention;

FIG. 2 illustrates an analysis of a sentence for annotation in accordance with one or more embodiments of the present invention;

FIG. 3 illustrates a flow diagram for a process for bootstrapping relation training data in accordance with one or more embodiments of the present invention;

FIG. 4 illustrates a cloud computing environment according to one or more embodiments of the present invention;

FIG. 5 illustrates abstraction model layers according to one or more embodiments of the present invention; and

FIG. 6 illustrates a block diagram of a computer system for use in implementing one or more embodiments of the present invention.

The diagrams depicted herein are illustrative. There can be many variations to the diagrams or the operations described therein without departing from the spirit of the invention. For instance, the actions can be performed in a differing order, or actions can be added, deleted, or modified. Also, the term “coupled” and variations thereof describes having a communications path between two elements and does not imply a direct connection between the elements with no intervening elements/connections between them. All of these variations are considered a part of the specification.

### DETAILED DESCRIPTION

One or more embodiments of the present invention provide computer-implemented methods, computing systems, and computer program products that generate relation annotations by analyzing co-occurring entities and any intervening tokens in document passages.

Training a model to detect relations in a document is a labor intensive task. Conventional methods typically begin with collecting documents from a corpus and manually reviewing the document's text. The text is manually annotated with labels that identify any entities in the documents. Once the entities are annotated, the entity labels are examined to determine if there are relations between entities. A set of annotated documents can be produced by one or more human annotators that are analyzing the corpus. The set of documents can be used as training data to train a relation model to later help automate the process. However, prior to annotation, a decision that needs to be addressed is the expertise of an annotator. In some instances, for example, the annotator can be one of the software developers designing the system, in another instance, the annotator can be a medical health professional. In any case, the decision on the annotator brings along each annotator's individual judgment. As a result, one annotator can choose to annotate a document passage differently than another annotator.

For example, the following sentence can be a passage found in a document, “The use of Aggrenox can sometimes lead to headaches, dyspepsia, stomach pains, nausea, and diarrhea”. Within this passage, an annotation for an entity describing “Aggrenox” can be “drug”. An annotation for an entity describing “ headaches, dyspepsia, stomach pains, nausea, and diarrhea” can be “symptoms”. An annotator can review the passage on a computer and select the entities “drug” and “symptoms,” and the system will produce a set of annotations from an ontology that potentially describe a relation between these two co-occurring entities. The annotator can select one or more relation annotations to describe a relation between the entities. For example, the annotator can select the annotation “CanCause”. This annotated passage can later become part of a training data set. However, the choice of annotations is dictated by the multiple annotators working to build a training set. The annotator's work is not reviewed after the annotations are complete. Therefore, it is difficult to ascertain the value of the training set in terms of adequately training a relation model.

One or more embodiments of the present invention address one or more of the above-described shortcomings by providing computer-implemented methods, computing systems, and computer program products that generate relation annotations by analyzing co-occurring entities and any intervening tokens in document passages.

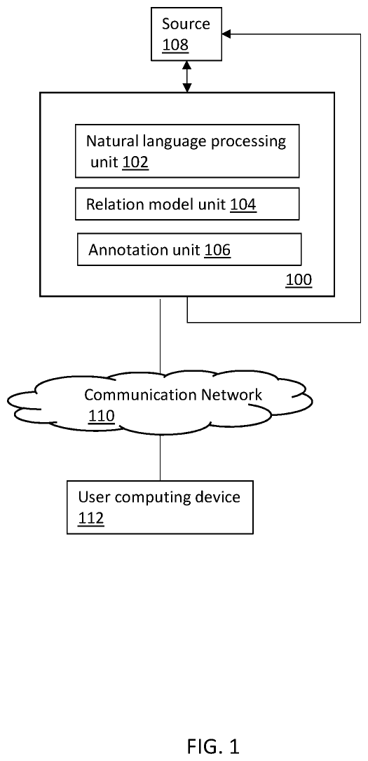

Turning now to FIG. 1, a system 100 for bootstrapping relation training data is generally shown in accordance with one or more embodiments of the present invention. The system 100 includes a natural language processing unit 102 for analyzing passages of a document to determine whether identified co-occurring entities have a desired relation type. The system 100 further includes a relation model unit 104 for analyzing the documents in a corpus to determine if passages include entities having the desired relation type, in which co-occurring entities are not identified. The system 100 further includes an annotation unit 106 for labeling the unidentified entities and labeling the relation between the entities. The annotated documents can be used as training data for training a relation model.

The natural language processing unit 102 is operable to receive data from a corpus for the analysis of relations between entities found in the data. The natural language processing unit 102 uses machine learning algorithms to analyze passages in the document for co-occurring entities. Co-occurring entities are at least two entities that occur within the same passage. The natural language processing unit 102 can further analyze intervening tokens between the co-occurring entities to determine if there is any congruency between the relation of the co-occurring entities and a desired relation type. The intervening tokens are words that connect the co-occurring entities within the passage. The intervening tokens and the co-occurring entities are used to identify the subject and predicate of a passage. The subject and predicate and further be used to identify predicate frames (sometimes referred to as case frames) in the passage. Predicate frames are structures that define syntactic and semantic properties of a passage, including but not limited to the semantic function of any arguments, a number of arguments, and a syntactic category of a predicate. For example, a passage could be the sentence, “The doctor suggested that the patient take aspirin to reduce any pain and inflammation”. In this instance, the co-occurring entities include “aspirin”, which is a type of drug and “pain and inflammation”, which are types of symptoms. The intervening tokens in this instance are “to”, “reduce”, and “any”.

The natural language processing unit 102 can identify potential co-occurring entities based on a desired relationship. The desired relationship can be in the form of a set of words or relation types labels selected by a user. For example, the desired relation can be the words “may treat” or can be a relation label type. Relation labels are typically made up of conjoined words and natural language processing unit 102 can split to attain tokens or words we can match against the intervening tokens of entities co-occurring within a sentence (e.g., “mayTreat” becomes “may treat”). In either instance, the natural language processing unit 102 can identify the verb “treat”. Co-occurring identities are typically defined in the context of relationship frames and semantic/thematic slots. A verb usually encodes a relationship between entities and the corresponding semantic frame defines the semantic slots allowed in that frame. These slots are the entities and are used to determine co-occurrence. For example, a passage can be “Aspirin may treat a headache”. The verb frame ‘treat’ requires an agent giving the approval, and in this example, the agent could be “Aspirin”. The verb frame also requires a theme, i.e. something that can be treated, and in this example, the theme can be “headache”. The natural language processing unit 102 can identify “aspirin” and “headache” as potential co-occurring identities.

The natural language processing unit 102 can analyze other passage to find the potential co-occurring identities “aspirin” and “headache”. The natural language processing unit 102 can further employ similarity techniques, such as word mover's distance (WMD) or cosine similarity to find other potential co-occurring entities that are similar in meaning to “aspirin” or “headache”. The similarity can be based on a word vector representing a potential co-occurring identity being a threshold value or a threshold difference from the identified potential co-occurring entities (e.g., “aspirin” or “headache”). The natural language processing unit 102 can further expand potential matches by using derivational morphology to analyze the relation (e.g., “treat” is related to “treatment”). Therefore if the intervening tokens between two potential co-occurring entities contain phrases such as ‘for the treatment of’ it could be considered a potential match for “mayTreat.” This would include potential co-occurring entities that are not similar to “aspirin” or “headache”.

Congruency is the compatibility between the relation described by the intervening tokens and the desired relation type. For example, if the desired relation type is one entity being a treatment for another entity, the intervening tokens, “to reduce any”, show that the co-occurring entities have the desired relation type. If, however, the desired relation type was that one entity is a side effect of another entity, the intervening tokens would demonstrate the co-occurring entities do not have congruency with the desired relation type. In terms of two phrases, congruency is having a similar or same semantic structure or meaning the same. For example, the phrases “is prescribed for the treatment of” and “is prescribed for” have a same semantic structure or same meaning.

The natural language processing unit 102 can semantically analyze the co-occurring entities and the intervening tokens to calculate a congruency score. Semantic analysis refers to measuring contextual similarity between words and phrases in a passage. The natural language processing unit 102 can determine a congruency score between a desired relation type and the words and phrases in the co-occurring entities and the intervening tokens. The calculated congruency score can be compared to a pre-defined threshold to either confirm or reject the relation between the co-occurring entities as a desired. If confirmed, the natural language processing unit 102 can annotate the co-occurring entities with a label describing the desired relation type.

The congruency score can be calculated using various techniques that produce a mathematical score and compare the score to a threshold value. For example, the natural language processing unit 102 can employ a cosine similarity technique or a word mover's distance (WMD) technique. The natural language processing unit 102 can convert the desired relation type and the words and phrases in the co-occurring entities and the intervening tokens in word vectors. The natural language processing unit 102 can further calculate a distance or score between the desired relation type and the words and phrases in the co-occurring entities and the intervening tokens. In the case of a WMD technique, a smaller score represents a shorter word embedding distance between the desired relation type and the words and phrases in the co-occurring entities and the intervening tokens. The natural language processing unit 102 can compare the score to a threshold value. If the score is below the threshold value, the natural language processing unit 102 can confirm the relation between the co-occurring entities. If the score is above the threshold value, the natural language processing unit 102 can reject the relation between the co-occurring entities.

The natural language processing unit 102 can aggregate each of the identified predicate frames. The natural language processing unit 102 can select keywords and phrases from these predicate frames to generate new words that have a similar or same meaning as the keywords. Using the example above, the natural language processing unit 102 can select the keywords “aspirin”, reduce”, and “pain and inflammation” as keywords and not consider “to” and “any”. In some embodiments of the present invention, the natural language processing unit 102 can employ a word embedding model to map the keywords and phrases, to respective numerical representations(e.g., word vectors). The natural language processing unit 102 can further search a domain-specific thesaurus and search query logs to find similar words and phrases. The natural language processing unit 102 can select on the domain-specific thesaurus based on the desired relation type. Based on a statistical similarity between numerical representations of the keywords and the retrieved words being greater than a threshold value, the natural language processing unit 102 can add the similar words and phrases to the aggregated set of co-occurring entities and intervening tokens.

The relation model unit 104 can further receive the expanded list of keywords and phrases and reanalyze the documents in a corpus. The relation model unit 104 can use machine learning algorithms to reanalyze documents in the corpus. The relation model unit 104 can analyze the document for additional passages that include the desired relation type, but do not include previously identified relevant co-occurring identifies. The relation model unit 104 can use the expanded list of keywords to semantically reanalyze the documents in the corpus 108. The relation model unit 104 can further detect predicate frames that are congruent with the previously identified predicate frames. The relation model unit 104 can calculate a congruency score between the identified predicate frame with the predicate frames identified by the natural language processing unit 102. The predicate frames identified by the relation model unit 104 do not need to include co-occurring entities that were identified by the natural language processing unit 104.

In some embodiments of the present invention, the relation model unit 104 can index the documents in terms of keywords. The relation model unit 104 can search the index as opposed to search a plurality of documents in the corpus 108. The relation model unit 104 can map the indexed keywords to documents in the corpus 108. Once a keyword is detected, the relation model unit 104 can follow the mapping to the document. Therefore, rather than a document-based search, the relation model unit can perform a keyword-based search. This method increases the speed at which documents can be retrieved from the corpus 108.

The relation model unit 104 can use semantic analysis to account for missing or misspelled words that caused the natural language unit 102 to not find co-occurring entities. For example, the natural language processing unit 102 may have identified “cisplatin” as an entity potentially relevant to the desired relation type of “TreatmentFor”. However, a passage may have included a misspelled version of “cisplatin” or may include a novel drug, “drugX”, for the treatment of lung cancer. In each of these instances, the natural language processing unit 102 may not have identified the passage as containing a relevant relation and not further analyzed. However, the relation model unit 104 can identify relevant predicate frames based on a congruency score. Based on a finding of a congruent predicate frame in a passage, the relation model unit 104 can further determine that the passage contains co-occurring entities. The relation model unit 104 can semantically and syntactically analyze the passage to detect the co-occurring entities regardless of the misspelling. For example, a passage may read, “Charles was prescribed sisplatine for treatment of lung cancer”. The relation model unit 104 can determine that the passage contains a predicate frame that is congruent with a predicate frame identified by the natural language processing unit 102. This identification is in spite of the word “cisplatin” being misspelled “sisplatine”. The relation model 104 can then semantically and syntactically analyze the passage and determine that “sispaltine” and “lung cancer” are co-occurring entities that are relevant to desired relation type.

The annotation unit 106 is operable to annotate the entities and relations in the passages identified as relevant by the relation model unit 104. The annotation unit 106 can label tokens to describe an entity type. The annotation unit 106 can determine the entity type, based on an analysis of a passage identified by the natural language processing unit 102 and a congruent passage identified by the relation model unit 104. For example, the natural language processing unit 102 can identify the first passage, “Patient was prescribed cisplatin for the treatment of lung cancer” as relevant to the desired relation type. The natural language processing unit 102 can further detect metadata describing the entity type of “cisplatin” as “drug” and the entity type of “lung cancer” as “medical condition”. The natural language processing unit 102 can have labeled the relation between “cisplatin” and “lung cancer” as “TreatmentFor”. The relation model unit 104 can have identified a second passage “Patent was prescribed sisplatine for the treatment of lung disease” as having a congruent predicate frame as the first passage. Based on a semantic analysis, the relation model 104 can determine that “sisplatine” and “lung disease” are relevant co-occurring entities. The annotation unit 106 is can further convert the co-occurring entities in the first and second passages to numerical representations (e.g., word vectors) and determine which co-occurring entity has a numerical representation within a threshold value of a numerical representation of another co-occurring entity. Based on analysis, the annotation unit 106 can conclude that and entity type for “cisplatin” is the same as an entity type for “sisplatine” and entity type for “lung cancer” is the same as an entity type for “lung disease”. The annotation unit 106 can annotate “sisplatine” with the label “drug”, and annotate “lung disease” with the label “medical condition”. The annotation unit 106 can further annotate the co-occurring entities “sisplatine” and lung disease” as having the relation “TreatmentFor”. The annotation passage can be used as training data to train a relation model, including the relation model used by the relation model unit 104.

The system is operable for receiving electronic data from a corpus 108. The corpus 108 can include a corpus, for example, the Unified Medical Language System (UMLS), the corpus 108 can also include electronic medical records from a health care provider, or other appropriate source of electronic documents. The data can include a document, which includes passages, such as topics, paragraphs, sentences, bullet points, and other logical units within a document. The passages can be annotated with labels that are metadata that describe a respective entity type for different tokens found in the passages. In some embodiments of the present invention, the document annotation can be performed prior to receipt by the natural language processing unit 102. In other embodiments of the present invention, the natural language processing unit 102 performs the entity annotation by applying an entity model.

The system 100 can be in operable communication with a user computing device 112 via a communication network 110. The system 100 can connect to the communication network via a communications port, a wired transceiver, a wireless transceiver, and/or a network card. The communication network 110 can transmit data using technologies such as Ethernet, fiber optics, microwave, xDSL (Digital Subscriber Line), Wireless Local Area Network (WLAN) technology, wireless cellular technology, 5G, Bluetooth technology and/or any other appropriate technology.

The user computing device 112 can be a desktop, laptop, smartphone, or other computing device. A user can operate the user computing device 112 to select a desired entity relation for the natural language processing unit 102 and the relation model unit 104.

The phrases “neural network” and “machine learning” broadly describes a function of electronic systems that learn from data. A machine learning system, engine, or module can include a machine learning algorithm that can be trained, such as in an external cloud environment (e.g., the cloud computing environment 50), to learn functional relations between inputs and outputs that are currently unknown. In one or more embodiments, machine learning functionality can be implemented using a natural language processing unit 102 and the relation model unit 104, having the capability to be trained to perform a currently unknown function. In machine learning and cognitive science, neural networks are a family of statistical learning models inspired by the biological neural networks of animals, and in particular, the brain. Neural networks can be used to estimate or approximate systems and functions that depend on a large number of inputs.

The natural language processing unit 102 and the relation model unit 104 can be embodied as so-called “neuromorphic” systems of interconnected processor elements that act as simulated “neurons” and exchange “messages” between each other in the form of electronic signals. Similar to the so-called “plasticity” of synaptic neurotransmitter connections that carry messages between biological neurons, the connections in the machine learning unit 104 that carry electronic messages between simulated neurons are provided with numeric weights that correspond to the strength or weakness of a given connection. During training, The weights can be adjusted and tuned based on experience, making the natural language processing unit 102 and the relation model unit 104 adaptive to inputs and capable of learning. After being weighted and transformed by a function determined by the network's designer, the activation of these input neurons are then passed to other downstream neurons, which are often referred to as “hidden” neurons. This process is repeated until an output neuron is activated. The activated output neuron determines which character was read.

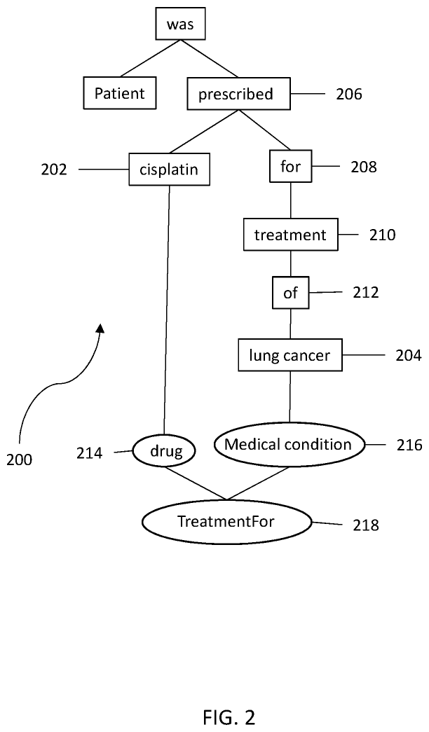

Referring to FIG. 2, an analysis of a parse tree 200 of a sentence for relation training data generation is illustrated. The natural language processing unit 102 and the machine learning unit 104 are trained to parse passages in a document into parse trees. In some embodiments of the present invention, natural language processing unit 102 and the machine learning unit 104 can convert each sentence into a parse tree. The term word refers to a syntactic word or, in other words a token. Each word or phrase in the sentence is a node of the parse tree. The parse tree includes a root node that is main verb of the sentence. Each node is connected by an edge indicating the direction of one word or phrase to another in the sentence. Each word or phrase is tagged with a label indicating a part of speech (e.g., verb, noun, adjective).

The parsed sentence illustrated in FIG. 2 would normally read, “ Patient was prescribed cisplatin for treatment of lung cancer”. “Cisplatin” 202 and “lung cancer” 204 are an example of co-occurring entities because they both appear within the same sentence boundary. The entity type for “cisplatin” 202, is “drug” 214. The entity type for “lung cancer” 204 is “medical condition” 216. The intervening tokens are for “cisplatin” 202 and “lung cancer” 204 are “prescribed” 206, “for” 208, “treatment” 210, and “of” 212. However, simply because these two entities are co-occurring entities, a relation between the co-occurring may not be a desired relation type. A determination can be made as to a desired relation type between the co-occurring entities. For example, the desired relation type between two entities can be “TreatmentFor” 218, or in other words, one entity is a treatment for another entity. In this sentence, “cisplatin” 202 is a treatment for “lung cancer” 204 and therefore, this sentence is a good sentence for a training set for detecting a treatment relation between two entities. It can be appreciated that another sentence could contain the words “cisplatin” and “lung cancer”, and the two words as written in the sentence not have the desired relation type of a drug that is a treatment for a medical condition.

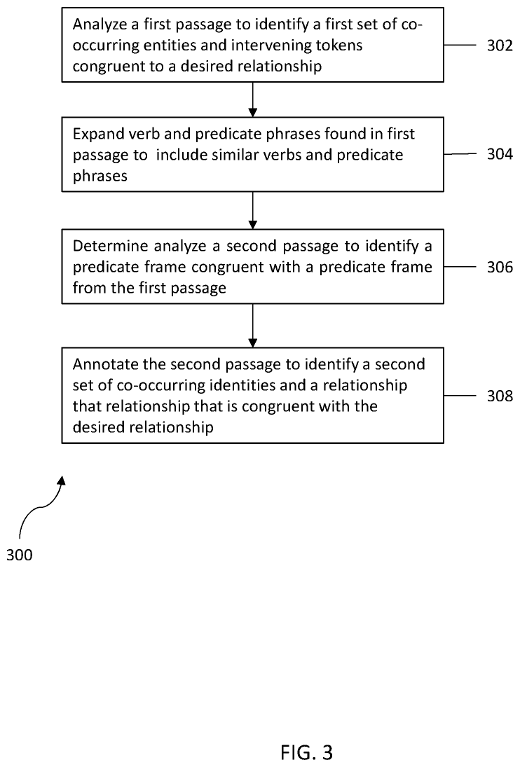

Referring to FIG. 3, a flow diagram 300 for a process for bootstrapping relation training data in accordance with embodiments of the present invention is shown. At block 302, a system can analyze a passage to identify co-occurring entities that are potentially relevant to a desired relation type. The passage can be a sentence, heading, bullet point, or other segment of an electronic document. Once the system identifies the co-occurring entities, it can identify any intervening tokens connecting the co-occurring entities. The co-occurring entities and the intervening tokens are analyzed to determine whether co-occurring entities are congruent with a desired relation type. If the co-occurring entities have the desired relation type. The system can further identify a predicate frame found in the passage. Each passage of each document in a corpus can similarly be traversed and analyzed for the identification of co-occurring entities that have the desired relation type.

At block 304, the system can expand the keywords found in the predicate frame found in the intervening tokens. The system can apply thesaurus, dictionary or other method to find words that have a similar meaning to keywords. To determine a similarity, the system can convert the keywords and potentially similar meaning words into respective numerical representations, for example, a word vector. The word vector is a numerical representation that describes a meaning of the word. The system can then determine whether a keyword has the same meaning as the other word. For example, a predicate frame may contain the verb “running” and system may consult a thesaurus and find the word “jogging”. The system can convert both word “running” and “jogging” into numerical representations. The system can further perform a statistical analysis to determine a similarity of the words based on the numerical representations. Based on the numerical representations, the words may be considered similar. However, the system can further perform a semantic analysis of the original sentence that produced the keyword “running”. For example, the sentence may be “I went running through the park.” In this instance, the term “jogging” can be considered to have a similar meaning. However, if the sentence was, “I accidentally left the car running”, the term “jogging” is not considered to have a similar meaning.

At block 306, the system can analyze a second passage to identify a predicate frame that is congruent with the predicate frame found in the first passage. However, the second passage does not include co-occurring entities that are identified as relevant to a desired relation type. This can be due to a grammatical error, an unidentified word, syntactically error, or other reasons. The system can further perform a semantic analysis to determine any similarities between the first passage and the second passage. For example, the system can determine whether any entities in the first passage are similar in meaning to any entities in the second passage.

At block 308, the system can annotate the entities in the second passage that were determined to be similar to the entities in the first passage. The system can detect an entity type label of a co-occurring entity from the first passage and label a similar entity in the second passage with the same entity type label. The system can further detect the relation type label between the co-occurring entities in the first passage, and label the co-occurring entities in the second passage with same relation type label.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

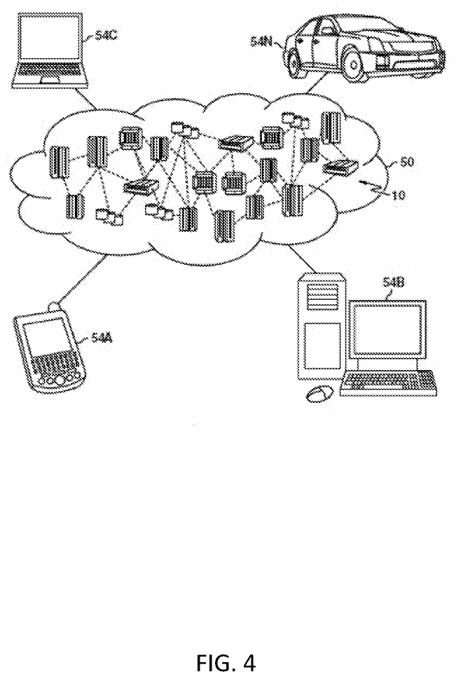

Referring now to FIG. 4, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

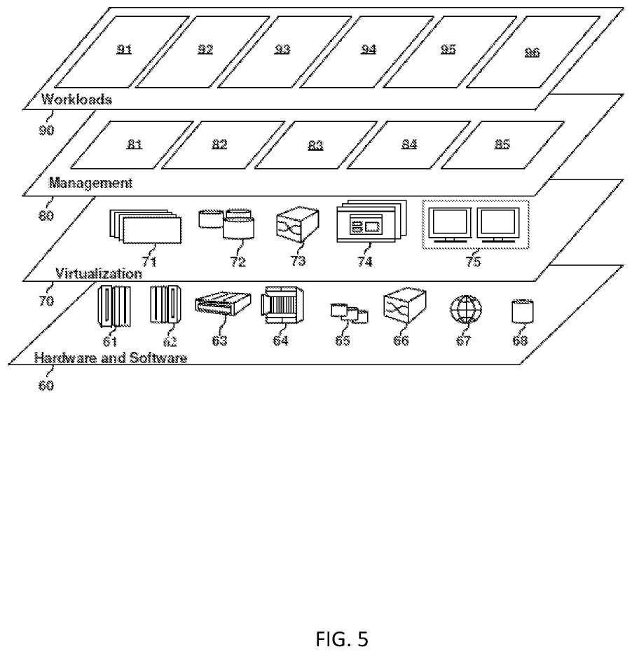

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and generating training data 96.

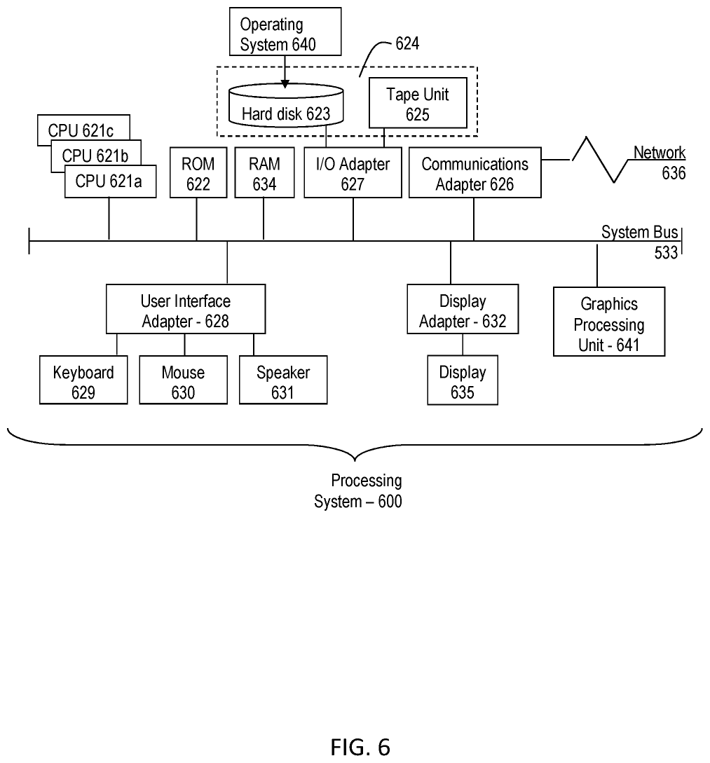

It is understood that the present disclosure is capable of being implemented in conjunction with any other type of computing environment now known or later developed. For example, FIG. 6 depicts a block diagram of a processing system 600 for implementing the techniques described herein. In examples, the processing system 600 has one or more central processing units (processors) 621a, 621b, 621c, etc. (collectively or generically referred to as processor(s) 621 and/or as processing device(s)). In aspects of the present disclosure, each processor 621 can include a reduced instruction set computer (RISC) microprocessor. Processors 621 are coupled to system memory (e.g., random access memory (RAM) 624) and various other components via a system bus 633. Read only memory (ROM) 622 is coupled to system bus 633 and may include a basic input/output system (BIOS), which controls certain basic functions of the processing system 600.

Further depicted are an input/output (I/O) adapter 627 and a network adapter 626 coupled to the system bus 633. I/O adapter 627 may be a small computer system interface (SCSI) adapter that communicates with a hard disk 623 and/or a storage device 625 or any other similar component. I/O adapter 627, hard disk 623, and storage device 625 are collectively referred to herein as mass storage 634. Operating system 640 for execution on processing system 600 may be stored in mass storage 634. The network adapter 626 interconnects system bus 633 with an outside network 636 enabling processing system 600 to communicate with other such systems.

A display (e.g., a display monitor) 635 is connected to the system bus 633 by display adapter 632, which may include a graphics adapter to improve the performance of graphics intensive applications and a video controller. In one aspect of the present disclosure, adapters 626, 627, and/or 632 may be connected to one or more I/O busses that are connected to the system bus 633 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 633 via user interface adapter 628 and display adapter 632. An input device 629 (e.g., a keyboard, a microphone, a touchscreen, etc.), an input pointer 630 (e.g., a mouse, trackpad, touchscreen, etc.), and/or a speaker 631 may be interconnected to system bus 633 via user interface adapter 628, which may include, for example, a Super I/O chip integrating multiple device adapters into a single integrated circuit.

In some aspects of the present disclosure, the processing system 600 includes a graphics processing unit 637. Graphics processing unit 637 is a specialized electronic circuit designed to manipulate and alter memory to accelerate the creation of images in a frame buffer intended for output to a display. In general, graphics processing unit 637 is very efficient at manipulating computer graphics and image processing and has a highly parallel structure that makes it more effective than general-purpose CPUs for algorithms where processing of large blocks of data is done in parallel.

Thus, as configured herein, the processing system 600 includes processing capability in the form of processors 621, storage capability including system memory (e.g., RAM 624), and mass storage 634, input means such as keyboard 629 and mouse 630, and output capability including speaker 631 and display 635. In some aspects of the present disclosure, a portion of system memory (e.g., RAM 624) and mass storage 634 collectively store the operating system 640 to coordinate the functions of the various components shown in the processing system 600.

Various embodiments of the invention are described herein with reference to the related drawings. Alternative embodiments of the invention can be devised without departing from the scope of this invention. Various connections and positional relations (e.g., over, below, adjacent, etc.) are set forth between elements in the following description and in the drawings. These connections and/or positional relations, unless specified otherwise, can be direct or indirect, and the present invention is not intended to be limiting in this respect. Accordingly, a coupling of entities can refer to either a direct or an indirect coupling, and a positional relation between entities can be a direct or indirect positional relation. Moreover, the various tasks and process steps described herein can be incorporated into a more comprehensive procedure or process having additional steps or functionality not described in detail herein.

One or more of the methods described herein can be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

In some embodiments, various functions or acts can take place at a given location and/or in connection with the operation of one or more apparatuses or systems. In some embodiments, a portion of a given function or act can be performed at a first device or location, and the remainder of the function or act can be performed at one or more additional devices or locations.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The present disclosure has been presented for purposes of illustration and description, but is not intended to be exhaustive or limited to the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

The diagrams depicted herein are illustrative. There can be many variations to the diagram or the steps (or operations) described therein without departing from the spirit of the disclosure. For instance, the actions can be performed in a differing order or actions can be added, deleted or modified. Also, the term “coupled” describes having a signal path between two elements and does not imply a direct connection between the elements with no intervening elements/connections therebetween. All of these variations are considered a part of the present disclosure.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or obj ect code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
traversing, by a processor for a first instance, a corpus to detect a first passage having a first set of co-occurring entities and intervening tokens associated with a desired relation type;
identifying, by the processor, a first predicate frame of the first passage based on the co-occurring entities and intervening tokens;
traversing, by the processor for a second instance, the corpus to detect a second passage having a second predicate frame with a same semantic structure as the first predicate frame, wherein the passage contains a second set of co-occurring entities associated with the desired relation type during first instance that the processor did not detect during the first instance;
detecting, by the processor, a second set of co-occurring entities in the second passage based on the second predicate frame; and
annotating, by the processor, the second set of co-occurring entities to have a same desired relation type as the first set of co-occurring entities.

2. The computer-implemented method of claim 1, wherein traversing the corpus to detect a second passage comprises:
extracting keywords from the first predicate frame;
converting each keyword into a keyword vector; and
detecting additional keywords based on the respective word vectors, wherein each additional keyword can be represented by a respective word vector having a value within a threshold value of the keyword vectors.

3. The computer-implemented method of claim 2, wherein traversing the corpus to detect a second passage comprises:
indexing each document in the corpus via an index keyword, wherein each index keyword is mapped to a document;
searching the index keywords for matching additional keywords;
retrieving a document from the corpus based on detecting an additional keyword.

4. The computer-implemented method of claim 2 further comprising:
selecting a domain-specific thesaurus based on the desired relation type between co-occurring entities; and
detecting the additional keywords from the domain specific thesaurus.

5. The computer-implemented method of claim 1 further comprising annotating the each entity of the second set of co-occurring entities to have a same entity type as a respective entity of the first set of co-occurring entities.

6. The computer-implemented method of claim 1, wherein the corpus is a medical based corpus.

7. The computer-implemented method of claim 1 further comprising:
training a relation model to detect co-occurring entities having a desired relation type using the second passage.

8. A system comprising:
a memory having computer readable instructions; and
one or more processors for executing the computer readable instructions, the computer readable instructions controlling the one or more processors to perform operations comprising:
traversing, by a processor for a first instance, a corpus to detect a first passage having a first set of co-occurring entities and intervening tokens associated with a desired relation type;
identifying, by the processor, a first predicate frame of the first passage based on the co-occurring entities and intervening tokens;
traversing, by the processor for a second instance, the corpus to detect a second passage having a second predicate frame with a same semantic structure as the first predicate frame, wherein the passage contains a second set of co-occurring entities associated with the desired relation type during first instance that the processor did not detect during the first instance;
detecting, by the processor, a second set of co-occurring entities in the second passage based on the second predicate frame; and
annotating, by the processor, the second set of co-occurring entities to have a same desired relation type as the first set of co-occurring entities.

9. The system of claim 8, wherein traversing the corpus to detect a second passage comprises:
extracting keywords from the first predicate frame;
converting each keyword into a word vector; and
detecting additional keywords based on the respective word vectors, wherein each additional keyword can be represented by a respective word vector having a value within a threshold value of the keyword vectors.

10. The system of claim 9, wherein traversing the corpus to detect a second passage comprises:
indexing each document in the corpus via a keyword, wherein each keyword is mapped to a document;
searching the index for additional keywords;
retrieving a document from the corpus based on detecting an additional keyword.

11. The system of claim 9, wherein the operations further comprise:
selecting a domain-specific thesaurus based on the desired relation type between co-occurring entities; and
detecting the additional keywords from the domain specific thesaurus.

12. The system of claim 8, wherein the operations further comprise annotating the each entity of the second set of co-occurring entities to have a same entity type as a respective entity of the first set of co-occurring entities.

13. The system of claim 8, wherein the corpus is a medical based corpus.

14. The system of claim 8, wherein the operations further comprise:
training a relation model to detect co-occurring entities having a desired relation type using the second passage.

15. A computer program product comprising a computer readable storage medium having program instructions embodied therewith, the program instructions executable by a processor to cause the processor to perform operations comprising:
traversing, by a processor for a first instance, a corpus to detect a first passage having a first set of co-occurring entities and intervening tokens associated with a desired relation type;
identifying, by the processor, a first predicate frame of the first passage based on the co-occurring entities and intervening tokens;
traversing, by the processor for a second instance, the corpus to detect a second passage having a second predicate frame with a same semantic structure as the first predicate frame, wherein the passage contains a second set of co-occurring entities associated with the desired relation type during first instance that the processor did not detect during the first instance;
detecting, by the processor, a second set of co-occurring entities in the second passage based on the second predicate frame; and
annotating, by the processor, the second set of co-occurring entities to have a same desired relation type as the first set of co-occurring entities.

16. The computer program product of claim 15, wherein traversing the corpus to detect a second passage comprises:
extracting keywords from the first predicate frame;
converting each keyword into a word vector; and
detecting additional keywords based on the respective word vectors, wherein each additional keyword can be represented by a respective word vector having a value within a threshold value of the keyword vectors.

17. The computer program product of claim 16, wherein traversing the corpus to detect a second passage comprises:
indexing each document in the corpus via a keyword, wherein each keyword is mapped to a document;
searching the index for additional keywords;
retrieving a document from the corpus based on detecting an additional keyword.

18. The computer program product of claim 16, wherein the operations further comprise:
selecting a domain-specific thesaurus based on the desired relation type between co-occurring entities; and
detecting the additional keywords from the domain specific thesaurus.

19. The computer program product of claim 15, wherein the operations further comprise annotating the each entity of the second set of co-occurring entities to have a same entity type as a respective entity of the first set of co-occurring entities.

20. The computer program product of claim 15, wherein the corpus is a medical based corpus.
